(19) United States
(12) Patent Application Publication
(10) Pub. No.: US 2002/0173695 A1
(43) Pub. Date: Nov. 21, 2002

(54) Physiologically-based control system and method for using the same

(76) Inventors: Mikhail Skliar, Salt Lake City, UT (US); Guruprasad A. Giridharan, Salt Lake City, UT (US)

Correspondence Address: KENNETH E. HORTON, RADER, FISHMAN & GRAUER PLLC, RIVERPARK CORPORATE CENTER ONE, 10653 SOUTH RIVERFRONT PARKWAY, SUITE 150, SOUTH JORDAN, UT, 84095, US

(21) Appl. No.: 10/147,259

(22) Filed: May 15, 2002

Related U.S. Application Data
(60) Provisional application No. 60/291,363, filed on May 16, 2001.

(51) Int. Cl. A61N 1/362

(52) U.S. Cl. 600/16

(57) ABSTRACT

A device and method for maintaining a constant average pressure difference between the inlet and outlet of a pump for a body fluid, leading to an adequate flow for different pathological conditions. The device and method allow for automatic adjustment of the pump to meet the physiological demand of the patient. The device and method also allow the physiological constraints on the pump to be accounted for, preventing suction and minimizing back flow of the body fluid. The device and method allow implicit synchronization of the pump with the natural regulatory mechanism for meeting patient's demand. Thus, the pump can be continually adjusted to an optimal level in response to the patient's physiological condition.

### REFERENCE TO RELATED APPLICATION

This application claims priority from U.S. Provisional Patent Application No. 60/291,363, the entire disclosure of which is incorporated herein by reference.

### FIELD OF THE INVENTION

The invention relates to control systems for pumps for body fluids and methods for using such controllers. Specifically, the invention relates to a controller for continuously-driven blood pumps that automatically regulates the pump in accordance with the physiological needs of the patient. Even more specifically, the invention relates to a controller for continuously-driven heat pumps that automatically regulates the pump in accordance with the physiological needs of the patient.

### BACKGROUND OF THE INVENTION

Numerous types of pumps have been designed to help various parts of the body pump liquids, including the bladder, kidneys, and brain. See, for example, U.S. Pat. Nos. 4,554,069, 4,787,886, and 6,045,496, the disclosures of which are incorporated herein by reference. The primary use of such pumps have been to pump blood for the heart of a patient. See, for example, U.S. Pat. Nos. 4,509,946, 4,683,894, 4,648,877, 4,750,868, 5,007,927, 5,599,173, 5,807,737, 5,888,242, 5,964,694, 6,082,105, 6,135,943, 6,164,920, and 6,176,822, the disclosures of which are incorporated herein by reference

Blood pumps for assisting the heart have been—and are being developed—in a number of forms. One type of the heat blood pump is a ventricular assist device (VAD). VADs have been in use for many years as a bridge to transplantation and, therefore, hold a potential to become a long-term alternative to donor heart transplantation. There are numerous designs for pumps, but most suffer from serious problems, including wear, limited reliability and large size, and may cause hemolysis and thrombosis. One preferred type of VAD is an axial flow VAD, like the DeBakey/NASA left ventricular assist device (LVAD). The DeBakey/NASA LVAD is one of the smallest available in clinical trials, making it suitable for implantation even in smaller individuals and children. There are only two points of contact of moving parts to minimize wear in the DeBakey pump. The blood contact area is small and the surfaces are made of biocompatible highly polished titanium, which reduces the risk of blood damage and thrombus formation.

Currently, a control system for continuous flow VADs that automatically responds to physiological demand does not exist. The flow rate generated by continuous flow VAD, such as the DeBakey pump, is selected manually by a physician or other trained hospital personnel. Mobile patients can operate implanted continuous flow VADs in one of two ways: “automatic” and manual. During automatic control the patient, following guidelines provided by the doctor, manually sets the desired pump rpm depending on the level of physical activity. The VAD controller automatically adjusts the current and voltage applied to the pump, to achieve and maintain the desired rpm setpoint. No highly reliable feedback based on physiological measurements (such as pressures, flows, O₂ saturation, lactic acid concentration in blood, CO₂ pressure, etc . . . ) is available. In manual mode, the patient directly adjusts the pump rpm by “twisting the knob” until a perceived comfort level of perfusion is achieved.

One type of controller for VADs has recently been proposed. See Waters et al. “Motor Feedback Physiological Control for a Continuous Flow VAD” Artificial Organs 1999; 23(2) 480-486, the disclosure of which is incorporated herein by reference. Waters et. al. present a representative picture of the current state-of-the-art in developing an improved control system for continuous flow VADs. A Proportional-Integral (PI) control system was developed for a simple computer model of circulatory system. The assumptions made in this work are unrealistic, including continuous flow throughout the circulatory system, no heart valves and linear correlation between pump generated pressure difference, ΔP, and pump voltage, current, and rpm. As such, the proposed controller is not suitable to be used in patients and a more suitable—and realistic—type of controller needs to be developed.

### SUMMARY OF THE INVENTION

The invention provides a control system—including a controller—for continuous-flow body fluid pumps that automatically responds to physiological demand. The invention includes a model of the human circulatory system incorporating circulatory support by a continuous-flow pump and a feedback controller designed to maintain physiologically sufficient flow of the needed body fluid. The model combines a network type model of the circulatory system with a nonlinear dynamic model of the continuous-flow pump. The invention operates by maintaining a constant instantaneous or time average pressure difference between the inlet and outlet of the pump, leading to an adequate flow of the body fluid for different pathological conditions.

The invention allows automatic adjustment of the pump parameters via a control system to meet the physiological demand of the patient, preventing suction and minimizing back flow of the body fluid. The control system allows implicit synchronization with the natural regulatory mechanism for meeting patient's demand. Thus, the pump can be continually and automatically adjusted to an optimal level in response to the patient's physiological condition.

The invention includes a method for pumping a body fluid by providing a pump for the body fluid, determining the pressure differential across the pump, and maintaining the pressure differential at a substantially constant value. The invention also includes a method for controlling a pump for a body fluid by providing a pump for the body fluid, determining the pressure differential across the pump, and maintaining the pressure differential at a substantially constant value. The invention yet further includes a method for controlling a pump for a body fluid by providing a pump for the body fluid, determining the pressure differential across the pump, and maintaining the pressure differential at a substantially constant value, thereby providing an adequate flow of the body fluid under different pathological conditions. As well, the invention includes a method for controlling a pump for a body fluid by providing a pump for the body fluid, measuring the average pressure differential across the pump by using pressure sensors, and maintaining the average pressure differential at a substantially constant value, thereby providing an adequate flow of the body fluid under different pathological conditions.

The invention also includes a system for pumping a body fluid, the system containing a pump, means for determining the pressure differential across the pump, and means for maintaining the pressure differential at a substantially constant value. The invention also includes a system for pumping a body fluid, the system containing a pump and a control system for maintaining a physiologically-sufficient flow of the body fluid through the pump, the control system maintaining a pressure differential across the pump to ensure adequate flow despite the changing conditions of the body fluid. The invention further includes a system for a body fluid, the system containing a pumping system for pumping the body fluid and a control system for maintaining a physiologically-sufficient flow of the body fluid through the pumping system, the control system maintaining a pressure differential across the pumping system to ensure adequate flow despite the changing conditions of the body fluid.

### BRIEF DESCRIPTION OF THE DRAWINGS

FIGS. 1-26 are views of several aspects of the fluid systems and methods for using the same according to the invention, in which:

FIGS. 1-5 illustrate a model used in the fluid system in one aspect of the invention;

FIGS. 6-7 illustrate the conditions of a healthy heart in one aspect of the invention;

FIGS. 8-9 illustrate the conditions of a weakened heart in one aspect of the invention;

FIGS. 10-11 illustrate the conditions of a asystolic heart in one aspect of the invention;

FIGS. 12-13 illustrate the conditions of a healthy heart assisted with the fluid system in one aspect of the invention;

FIG. 14 depicts one aspect of the fluid system when assisting a healthy heart;

FIGS. 15-16 illustrate the conditions of a weakened heart assisted with the fluid system in one aspect of the invention;

FIG. 17 depicts one aspect of the fluid system when assisting a weakened heart;

FIGS. 18-19 illustrate the conditions of a asystolic heart assisted with the fluid system in one aspect of the invention;

FIG. 20 depicts one aspect of the fluid system when assisting a asystolic heart;

FIGS. 21-22 illustrate the conditions of a weakened heart during exercise when assisted with the fluid system in one aspect of the invention;

FIG. 23 depicts one aspect of the fluid system when assisting a weakened heart during exercise;

FIGS. 24-25 illustrate the conditions of a weakened heart during exercise when assisted with the fluid system in one aspect of the invention; and

FIG. 26 shows some operating characteristics of the fluid system of the invention.

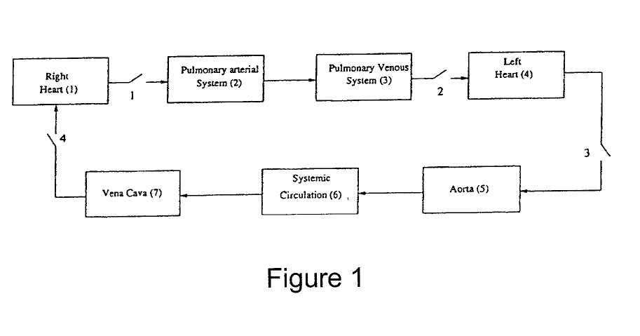

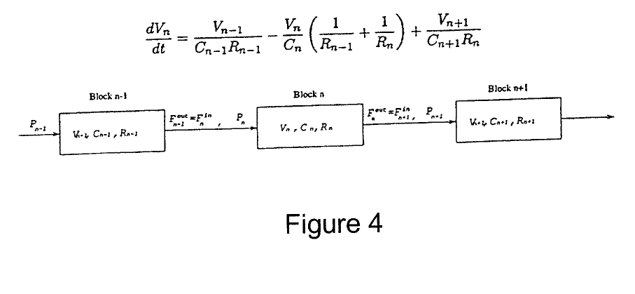

FIGS. 1-26 illustrate specific aspects of the invention and are a part of the specification. Together with the following description, the Figures demonstrate and explain the principles of the invention and are views of only particular—rather than complete—portions of the invention.

### DETAILED DESCRIPTION OF THE INVENTION

The following description provides specific details in order to provide a thorough understanding of the invention. The skilled artisan, however, would understand that the invention can be practiced without employing these specific details. Indeed, the present invention can be practiced by modifying the illustrated system and method and can be used in conjunction with apparatus and techniques conventionally used in the industry. For example, the invention is described below for pumps used in pumping blood for the heart, but could be modified for other body fluids and other body parts that pump liquids, including the bladder, kidneys, heart-lung machines and intravascular blood pumps.

The invention includes a fluid system for pumping body fluids and method for using the same. The fluid system comprises a pump system, a control system for controlling the pump system, and any other necessary components for the fluid system to operate. The pump system contains a pump and devices associated with operating the pump. The control system contains a controller for controlling or regulating the pumping system and any other devices associated with regulating the pump. The system of the invention is used, for example, to pump body fluids in a controlled manner.

In one aspect of the invention, the fluid system of the invention is used to pump blood through the heart. In this aspect of the invention, the pumping system contains any blood pump that is conventionally used, e.g., a VAD. In this aspect of the invention, the pumping system also contains components or devices—such as tubing, connectors, valves, sensors, power supply, and/or the like—that are typically used with the blood pump during its operation. See, for example, the description of such components or devices in U.S. Pat. Nos. 5,888,242, 4,683,894, 4,509,946, 4,787,886, 5,007,927, 5,599,173, 5,807,737, and 6,045,496, as well as European Patent Application No. 0503839A2, the disclosures of which are incorporated herein by reference.

In this aspect of the invention, the control system contains any suitable controllers that functions to regulate or control the pumping system. Examples of suitable controllers include those described below. In this aspect of the invention, the control system also contains components or devices—such as sensors, a monitoring system, and/or a feedback system—that are typically used with the controller during its operation. See, for example, the description of such components or devices in U.S. Pat. Nos. 5,888,242, 4,683,894, 4,509,946, 4,787,886, 5,007,927, 5,599,173, 5,807,737, and 6,045,496, as well as European Patent Application No. 0503839A2, the disclosures of which are incorporated herein by reference.

In one aspect of the invention, two pressure sensors and an rpm sensor were used to control the pump. In another aspect of the invention, however, the pressure sensors can be eliminated by using readily available measurements of the pump rpm, voltage, and current information to estimate the pressure differential between the left heart (LH) and the aorta. Eliminating the sensors leads to a controller that estimates the intrinsic pump parameters to control the LVAD, eliminating the need for pressure sensors and resulting in a simplified and more reliable control system.

In one aspect of the invention, the selected control objective of the control system for the blood pump is to maintain the pressure difference between the LH and the aorta close to the specified reference ΔP. The body maintains a constant average ΔP and varies the vascular resistances to maintain the required pressure and flow of blood. Maintaining the prescribed ΔP thus synchronizes the assist and natural perfusion, thereby incorporating natural cardiovascular regulation into the controller and allowing for simple control algorithms.

Thus, a PI controller was developed to vary the motor current of the blood pump to minimize the difference between the reference and the actual differential pressure when changes occurred in the circulatory system. One normal and two different pathological cases (second and third cases) were simulated to test the controller operation. In the first case, the VAD was attached to a normal healthy heart (as is the case following the recovery of natural LV function or during testing with animals). In the second case, the LVAD was attached to a weakened left heart and in the third case, the LVAD was attached to an asystolic left heart.

The design of the controller is broken into the selection of the control objectives, selecting the measurements (or control inputs) to be used in the feedback, and designing the control algorithms. The design process is iterative in nature, with the design step followed by performance evaluation that motivates the re-design goals. Thus, developing the model of the circulatory system is an integral step in designing the controller.

Selecting an adequate model for the controller avoids overwhelming complexity of the full-scale model of the entire circulation, but retains all relevant characteristics of that circulation model. However, unlike known controllers where a linear model with continuous flow throughout the system was assumed, the invention preserves such characteristics as nonlinearity, pulsatility, and discontinuity due to the effects of the natural heart valves.

The model for the controller design combines this circulation model (the model of the circulatory system) with a model of a continuous flow LVAD. The model subdivides the human circulatory system into an arbitrary number of lumped parameter blocks (or elements), each characterized by its own resistance, compliance, pressure and volume of blood. In one aspect of the invention, the model has eleven elements as illustrated in FIG. 1: 4 heart valves (1, 2, 3, 4), and 7 blocks including left heart (LH), right heart (RH), pulmonary and systemic circulation, vena cava and aorta. Hemodynamic details (such as the velocity profile) are not incorporated into the model, but can be if desired.

The detail of the model can be varied, e.g., increased or decreased. The detail can be increased by adding additional elements or by increasing the number of elements (or blocks). For example, the detail can be increased by subdividing the pulmonary and systemic circulations into constituent sub-elements. As well, the detail can be decreased by removing some of the elements or by reducing the number of elements by combining blocks together, e.g., by adding any of the blocks together.

Operation of each elements/block depends on its resistance (R) to the blood (of other body fluid) flow (F) and its compliance (C), which quantifies the ability of a given block to store a given blood volume (V). Two parameters, resistance and storage, are used to characterize each block. The storage element provides zero resistance to the flow, whereas the resistive element has zero volume. The resistance of an element or block is a function of the pressure drop and the blood flow across the block. The flow rate in and out of any block is a function of the pressure drop and resistance. The compliance is a function of the pressure and the stored volume of blood.

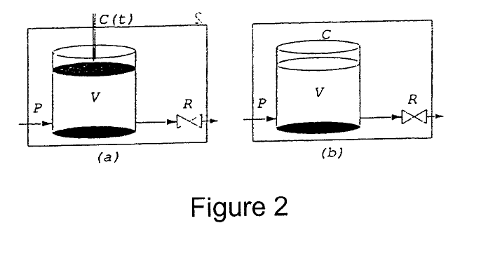

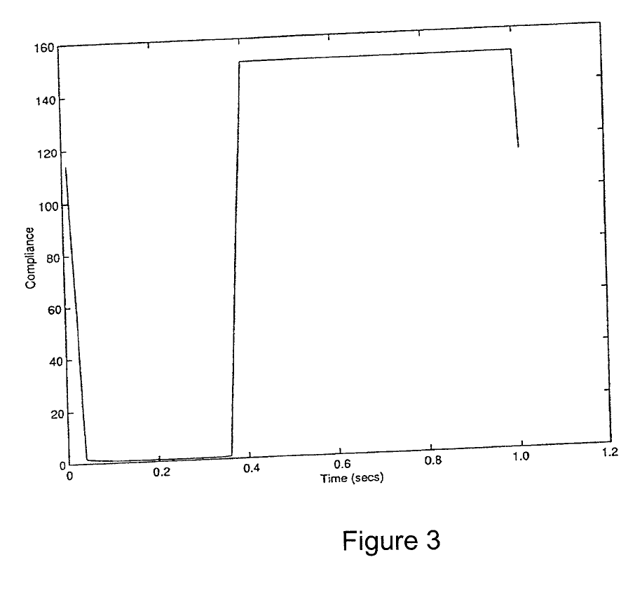

As illustrated in FIG. 2, each block can be categorized as passive or active. Active blocks represent heart chambers and are characterized by the varying compliance within each cardiac cycle. The rest of the blocks are passive. The varying compliance of the active blocks is responsible for the progression of the heartbeat. FIG. 3 illustrates an exemplary value of the compliance of an active block.

The volume of blood in any given block can be roughly described using a macroscopic material balance for that block. Accordingly, the volume of fluid for that block is a function of the resistance and compliance, which will differ in different patients and under different pathological conditions. In the invention, typical C and R values were assumed for all passive and active blocks and then were adjusted to reflect different pathological conditions under the three different cases.

The model includes four heart valves depicted in FIG. 1 as switches. A valve can be either fully open or fully closed. A valve is in open position when the upstream pressure is greater than the downstream pressure and is otherwise closed to prevent the back flow. In an open position, each valve has a finite and constant resistance to the blood flow. The resistance becomes infinite in the closed position. In other words, the heart valve is a block with no storage and with resistance that takes finite or infinite value depending on the sign of the differential pressure.

The model of the circulatory system is, therefore, a hybrid system that includes both dynamic and logical components. This circulatory model can be modified to include any desired pump and/or controller. In one aspect of the invention, the circulatory model is modified for an axial flow LVAD as an assist device. In this aspect of the invention, the VAD is driven by a brushless DC motor. A typical brushless DC motor is described in U.S. Pat. No. 5,888,242, the disclosure of which is incorporated herein by reference.

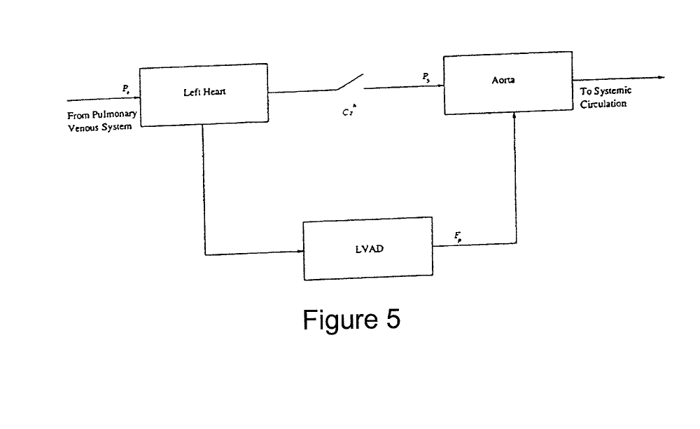

The model can be used for a pump system and control system used in parallel or in series with the heart. In one aspect of the invention, the model was designed for the case when the assist device works in parallel with the natural heart as depicted in FIG. 5. In this instance, the integration of the circulatory and LVAD models is simple and only affects the left heart and the aorta.

The rpm sensor can be integrated into the VAD design, as is the case with the DeBakey pump. However, measuring the differential pressure requires detecting the inlet and outlet pressures. In one aspect of the invention, two pressure sensors can be implanted for such detection. In another aspect of the invention, a sensorless VAD control system estimates the pressure differential by measuring the pump current I, voltage V, and rotational speed ω can be employed. See, for example, U.S. Pat. No. 6,135,944, the disclosure of which is incorporated herein by reference.

In the model, the compliances and resistances typically differ from patient to patient, and variations can occur for any given patient over any given time period. Since adaptive control strategies rely on using a nominal model with on-line adaptation, a multiple model adaptive approach as known in the art can be used to account for inter- and intra-patient variability in the circulatory system for better control.

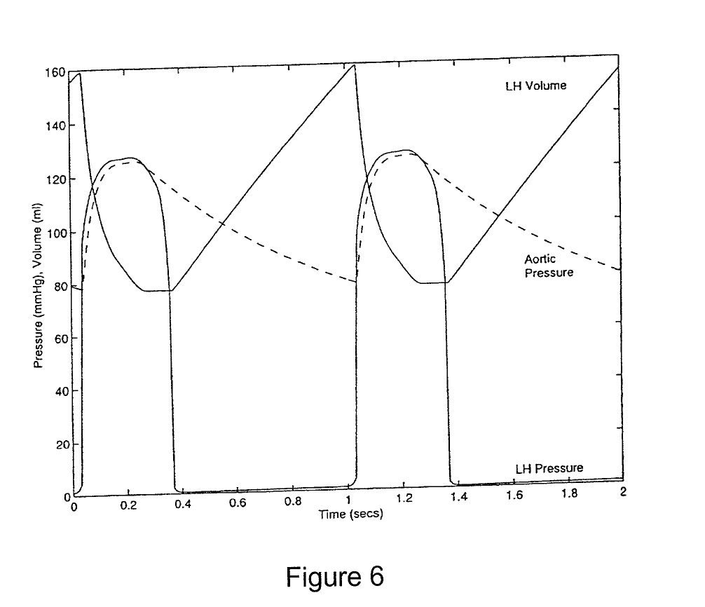

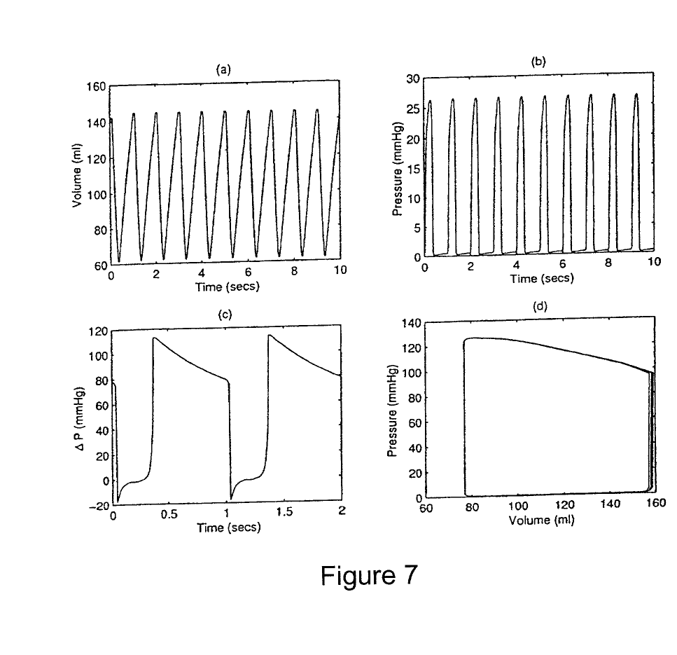

Using the model described above, three different cases with different conditions were simulated. The first case is the typical healthy heart whose characteristics are depicted in FIGS. 6 and 7. The stroke volume, which is the difference between the maximum and minimum volumes in the cardiac cycle, is 80 ml. The aortic systolic and diastolic pressures are 125/80 mmHg. There is a flow between the left heart (LH) and the aorta when the aortic pressure is lesser than the LH pressure. FIG. 7a shows the stroke volume of the right heart (RH) to be 80 ml, the same as the left heart. The RH peak pressure is 27 mmHg as seen from FIG. 7b. The normal range for RH pressure is 23-35 mmHg. The pressure difference between the aorta and the LH with progression of time is shown in FIG. 7c. The work done per stroke of the heart can be calculated using the area enclosed by the pressure-volume loop as illustrated in FIG. 7d.

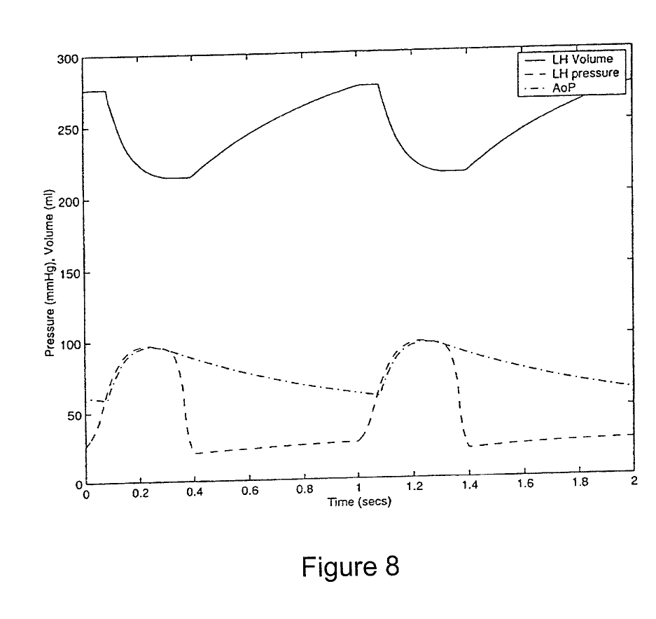

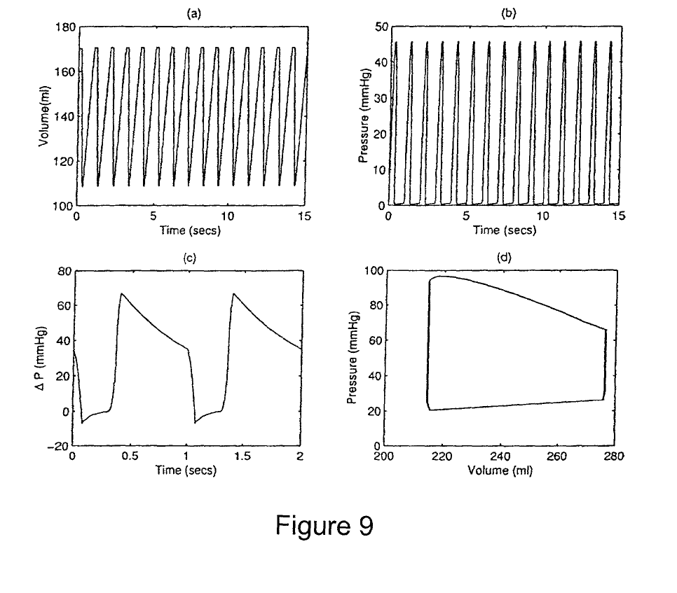

The second case is the failing heart whose characteristics are depicted in FIGS. 8 and 9. The failing heart has a lower stoke volume of approximately 60 ml and the aortic systolic and diastolic pressures are around 95/60 mmHg. A comparison with FIG. 6 shows that the LH volume is considerably higher than normal. The RH pressure is also much higher at 45 mmHg as depicted in FIG. 9b, which is typical for RH pressure with a failing left heart. Though not shown in figures, the simulation predicts edema in the pulmonary circulation, in the failing heart case. FIG. 9d shows that the work done by the weakened heart is less than the work done by the healthy heart, as the area of the pressure-volume loop is less than that of the normal heart.

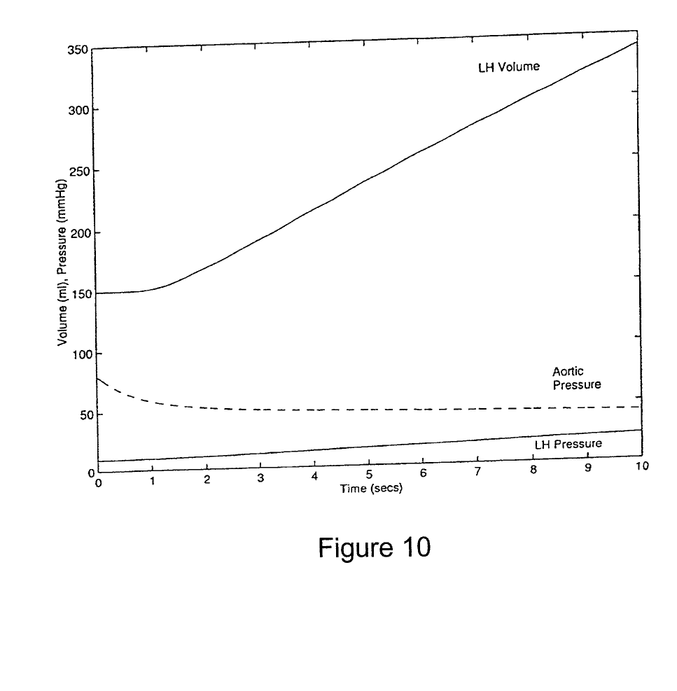

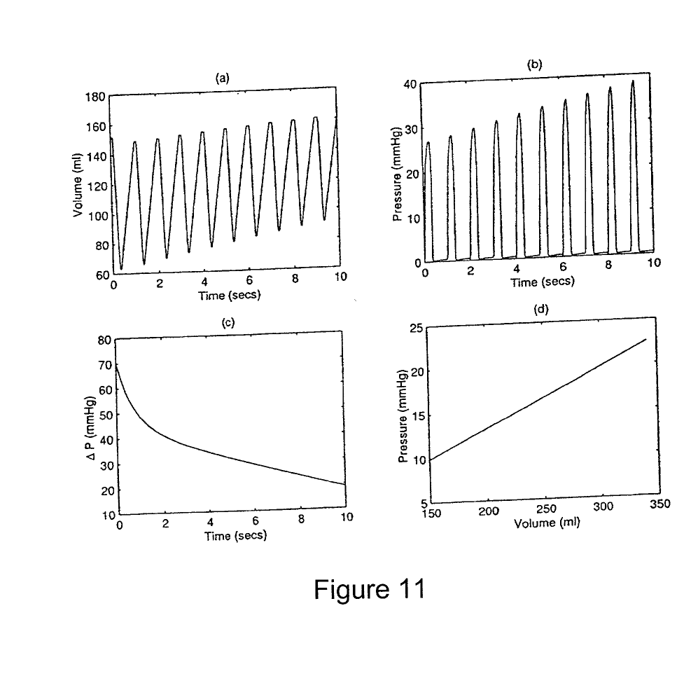

The third case is the asystolic LH heart whose characteristics are depicted in FIGS. 10 and 11. Asystole occurs to the whole heart, i.e. both LH and RH. The asystolic LH is used as an artifact to test the effectiveness of the PI controller. The LH volume should not rise above a certain value as the compliance for an asystolic LH heart decreases rapidly with increase in volume above a certain value. A constant compliance was assumed for the left heart for all LH volumes, resulting in the volume increase until about 1000 ml is reached when the physical forces equilibrate. This artifact does not affect the subsequent simulation with VAD feedback control since the controller is designed to keep the volume within narrow bounds, justifying the assumption of constant LH compliance. FIGS. 11a and 11b show an increasing RH volume and pressure. The absence of the pressure-volume loop in FIG. 11d indicates the terminal condition as the native heart produces no working stroke. FIG. 11c shows ΔP reducing constantly, indicating a sharp and definite decrease in circulation of blood. An asystolic left heart is the worst case for the LVAD load as it has to do all the work.

Based on this model, and the simulation in these three cases, the control system can be designed in the invention. The control system should function automatically. Further, the control system functions to adapt the VAD generated flow to the changing physiological requirements of the patient. Any control system meeting these requirements can be employed in the invention.

In one aspect of the invention, the control system maintains a constant instantaneous or time average pressure differential between the inlet and outlet of the pump. Maintaining a reference differential pressure is an effective way to the correct adaptation of the cardiac output to the changing requirements of the body because it is known that the vascular bed resistance can increase or decrease by a factor of 2 to 5. Since the blood flow is directly proportional to ΔP and inversely proportional to the vascular bed resistance, maintaining a constant ΔP with changing bed resistance can increase or decrease the flow rate by a factor of 2 to 5.

The reference ΔP can be maintained by adjusting the pump rpm. The pump rpm should be adjusted within physiologically admissible limits despite changing patient's vascular resistance, stroke volume, and pulse of the natural heart. All of these factors represent—as known in the art—the response to natural regulatory mechanisms to the changing physiological cardiac output demands. By maintaining the prescribed ΔP, the assist and natural perfusion can be synchronized, indirectly incorporating natural cardiovascular regulation into the VAD control. Controlling ΔP also leads to relatively simple control algorithms. Further, basing the control system on controlling ΔP minimizes the components: it requires implanting only pressure sensors or using a system where ΔP is estimated from the readily measurable characteristics of pump itself, such as voltage, current, and rpm.

An additional advantage for selecting ΔP as a feedback for the control system is that controlling ΔP can be used to ensure that the pump rpm is maintained within the physiological limitations. One extreme—collapse of the heart—establishes the physiological limit on the minimal volume of blood in the heart chamber, and can be translated into the constraints on the pump rotational speed as a function of blood volume. The back-flow to the heart—the other extreme—can be determined when the pump rotational speed drops below the lower limit, which depends on the vascular resistance and the varying compliance of the natural heart.

The control system of the invention includes a feedback system that regulates the pump rpm within physiologically acceptable constraints. The feedback system also helps minimize the difference between the reference and the actual ΔP. Since pulsing of the heart leads to periodic changes in ΔP, the control system also functions to keep oscillations of the pump rpm low. Thus, the control system increases the pump life and the comfort level of the patient.

The controller used in the invention must operate within the parameters of the model, e.g., it must maintain an adequate perfusion under the range of conditions a heart will operate. To estimate such conditions, the controller is used under the three different cases mentioned above: healthy heart, heart collapse due to the VAD suction, and left heart asystole, under conditions of rest and heavy exercise. To help determine the operating parameters of the control system, a fixed control structure was selected followed by selecting adjusting the operating parameters to satisfy the operating constraints.

To maintain the reference differential pressure, the controller manipulates the motor current of the pump. The controller manipulates the motor current until the desired trade-off between the speed of response and the rpm oscillations can be obtained.

The invention can be demonstrated by the following non-limiting Example.

### EXAMPLE

The fluid system of the invention with a LVAD containing a PI controller was tested under widely varying physiological conditions. The pulse rate was 60 beats per minute during rest, and 135 bpm during exercise. The LVAD parameters used in the simulation were the same as in Choi et al. “Modeling and Identification of an Axial flow Blood Pump” Proceedings of the 1997 American Control Conference 3714-3715 (June 1997), the disclosure of which is incorporated herein by reference.

Before t=0, an unassisted perfusion was simulated. At time t=0, arbitrarily selected as the end of the diastole, the LVAD assistance was initiated with the reference differential pressure of 75 mmHg sent to the designed PI controller. The initial flow rate and rpm were set to zero, causing a large initial back flow of blood.

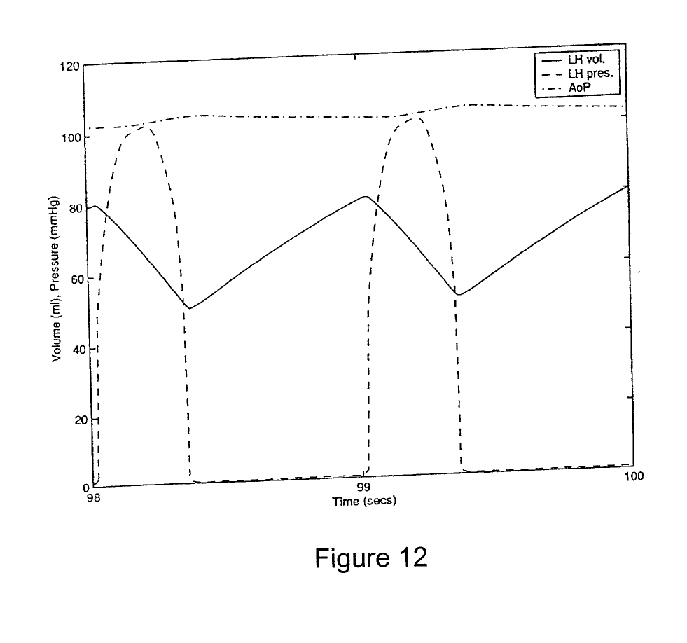

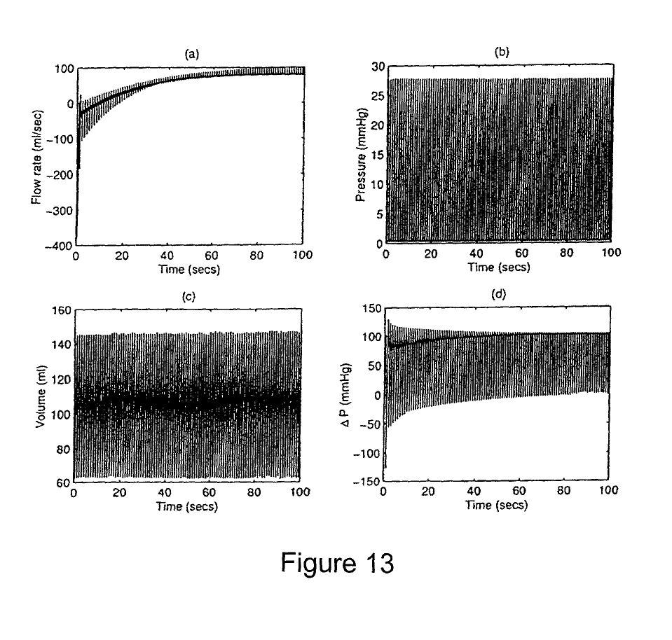

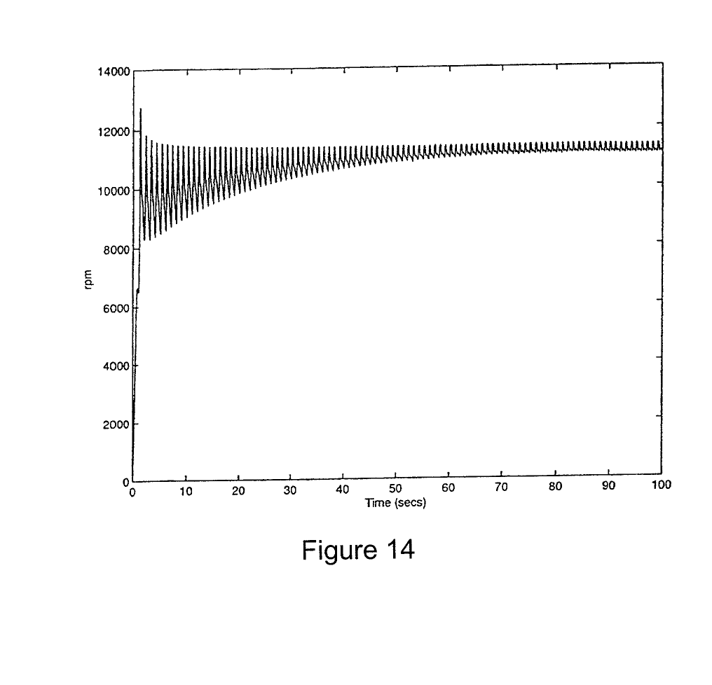

The LVAD and PI controller were first tested under a first case, the healthy heart. FIGS. 12, 13, and 14 show results for the healthy heart with VAD assistance. FIG. 12 indicates the reduction of the LH volume from about 70/150 ml observed without VAD to about 39/107 ml during LVAD operation. The minimum volume of 40 ml gave an adequate safety margin against suction of the LH. The aortic pressure was 121/89 mmHg with low pulsatility and the LH pressure changes from 0 to about 110 mmHg. As illustrated in FIG. 13a, the pump flow rate reached the limit cycle in less than 60 seconds. FIG. 13c shows almost the same stroke volume for the RH during the entire time. FIG. 13b shows the RH pressure, the maximum value of which is around 28 mmHg, well within the normal range. As depicted in FIG. 14, the rpm variations are reduced considerably after the initial transient period.

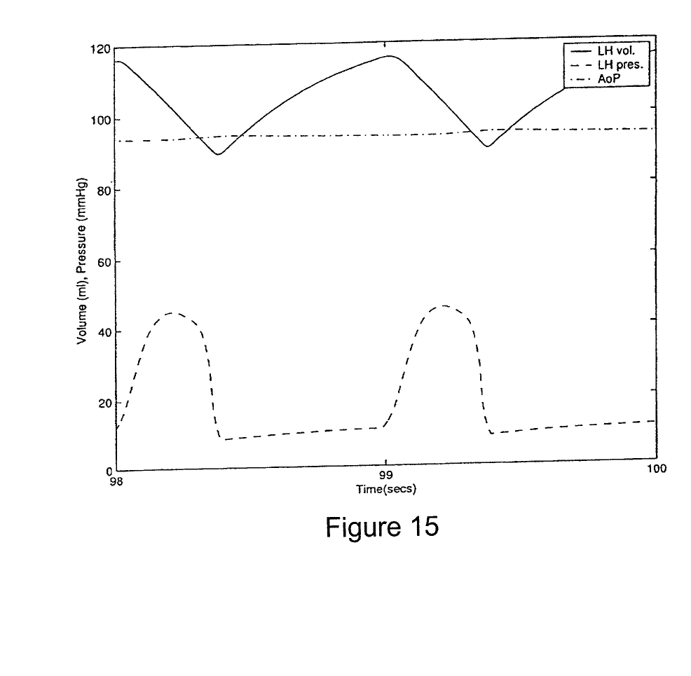

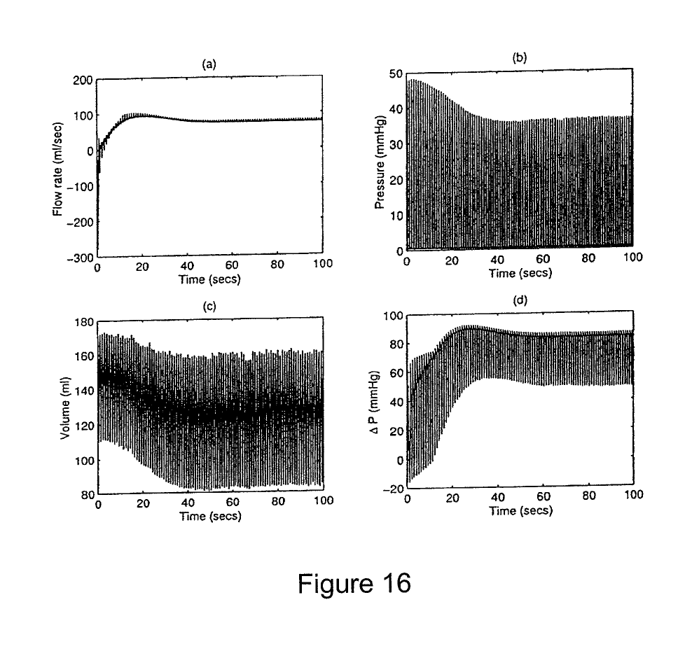

The LVAD and PI controller were next tested under the second case, the failing heart. FIGS. 15, 16, and 17 show the results of the simulation for the failing heart assisted by a VAD with the PI controller. FIG. 15 indicates a fairly constant aortic pressure of low pulsatility around 99/91 mmHg. The LH systolic and diastolic pressures are much closer to each other compared to a healthy heart with the LVAD. The volume of the LH with VAD support was reduced from 215/275 ml (without VAD assistance) to about 82/119 ml. The LH pressure was reduced to about 50/10 mmHg. As depicted in FIG. 16b, the RH pressure was reduced to around 35/0 mmHg, which is within the normal range. The lung edema also gradually reduced, indicating an adequate perfusion.

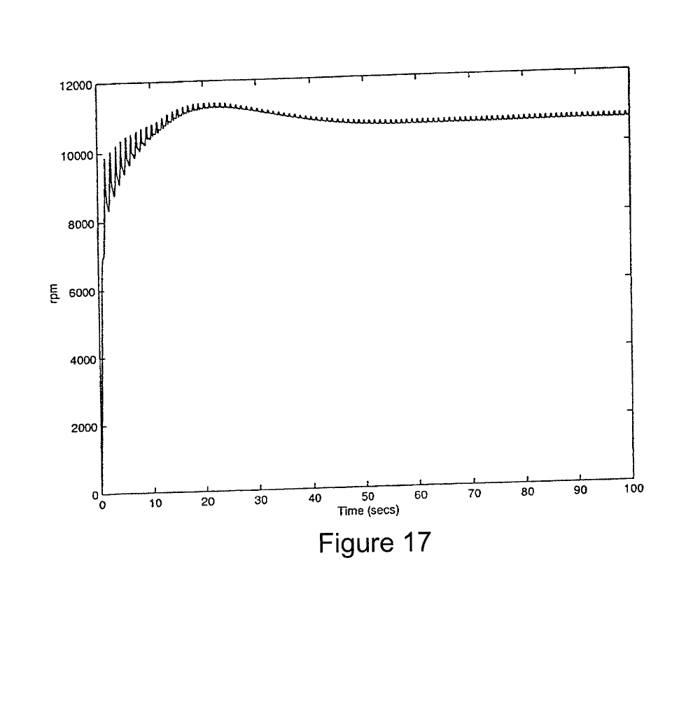

FIG. 16a shows that there was no back flow through the pump and also that the average pressure head closer to the 75 mmHg setpoint (as shown in FIG. 16d) compared to a weakened heart without a VAD. As illustrated in FIG. 16c, the stroke volume increased from 60 ml, a failure condition, to nearly 80 ml, the stroke volume for a normal heart as seen in FIG. 7. FIG. 17 shows that the rpm variations at the limit cycle were reduced and less than the rpm variations with a healthy heart. This was expected since the weakened heart is unable to produce the high pressure variations that are produced by a normal heart. The initial back flow of blood illustrated in FIG. 16 is due to the zero rpm starting condition.

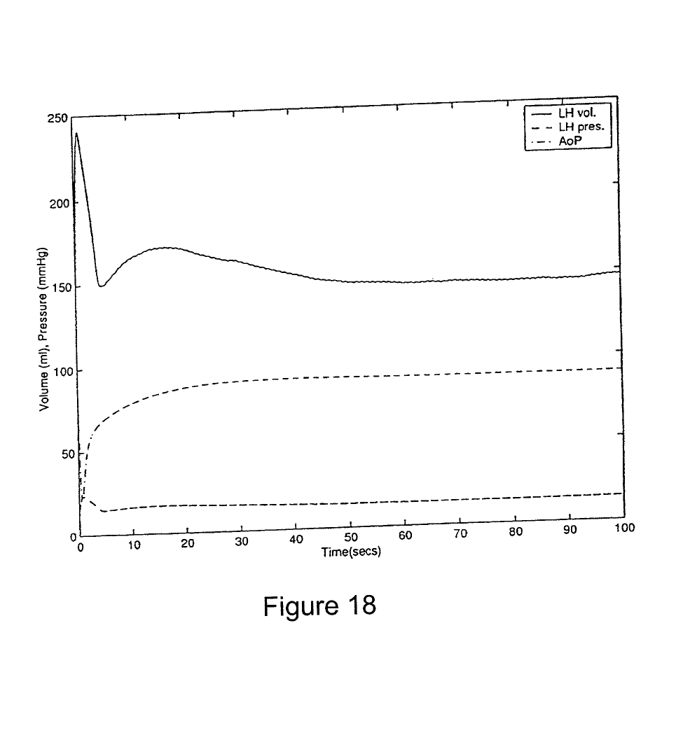

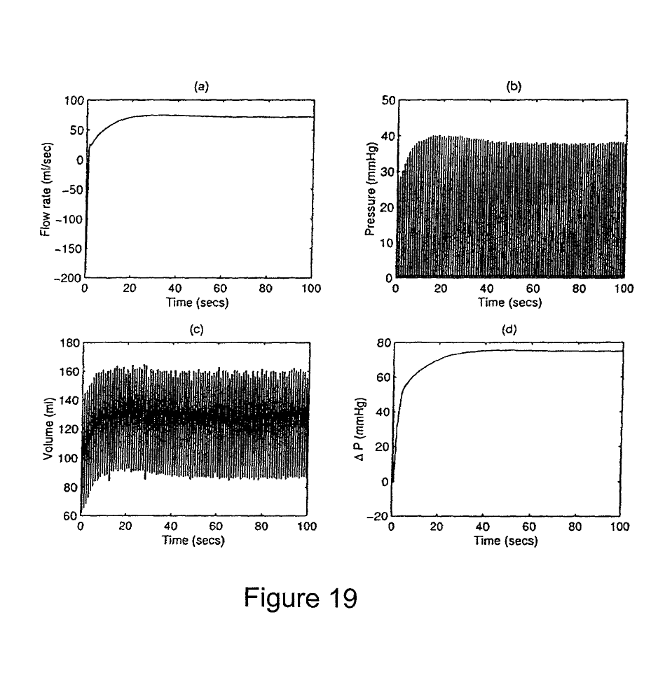

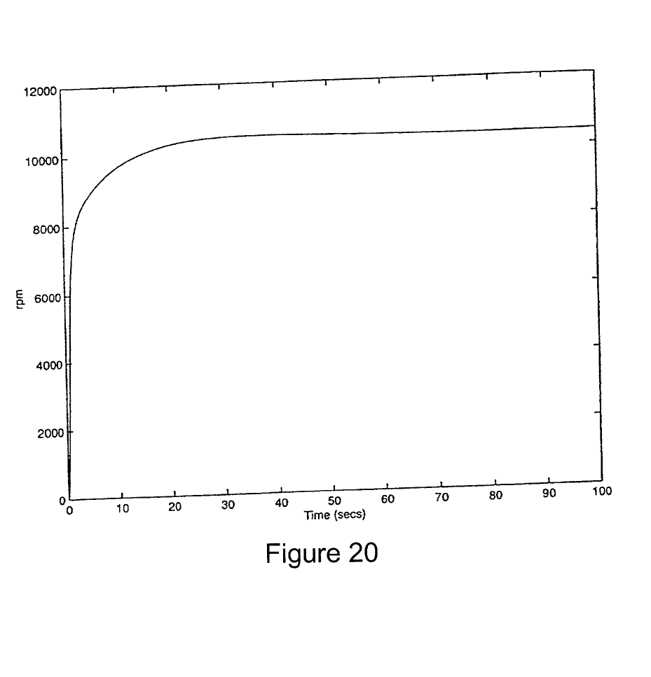

The LVAD and PI controller were finally tested under the case of an asystolic LH heart. FIGS. 18, 19, and 20 show the heart and VAD characteristics for an asystolic LH attached to a VAD. In FIGS. 18 and 19 the LH, and aorta volumes and pressures, pump flow rate, and pressure head settled to a single value after some initial oscillation. The RH volumes were stable and indicate adequate perfusion. The RH pressure stabilized at around 38/0, which is slightly elevated from the normal range despite a complete failure of the left heart. FIG. 20 shows that the rpm variations are absent as the asystolic LH does not produce any pressure variation.

Similar simulation studies were also performed for all three cases under heavy exercise. This was accomplished by reducing the time taken for each cardiac cycle and by altering the resistances for each block. The maximum factor by which the resistance was reduced was 3, as the pump flow rate exceeded 121 pm, the design limit for most of the axial flow blood pumps.

The cardiac demand during exercise was about triple the demand under rest. The minimum LH volume of 40 ml gave an adequate safety margin against ventricular collapse due to suction. The AoP remained constant at 109/98 mmHg and the systolic pressure of the LH was about 110 mmHg. The RH stroke volume was approximately 110 ml and the maximum RH pressure was 30 mmHg, indicating acceptable operating pressures and an adequate perfusion. There was an initial back flow of blood, due to the zero rpm starting condition. The pump had some rpm variation even after reaching the limit cycle due to the pulsatility of the heart.

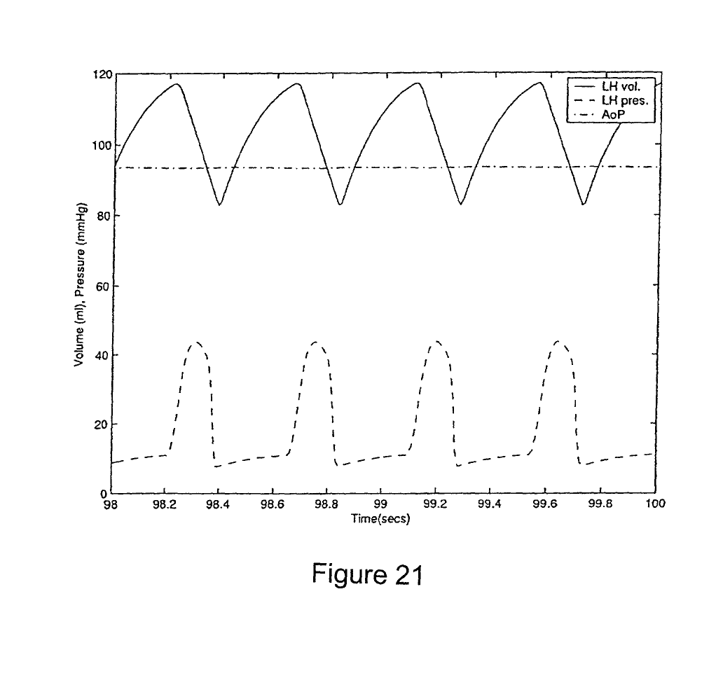

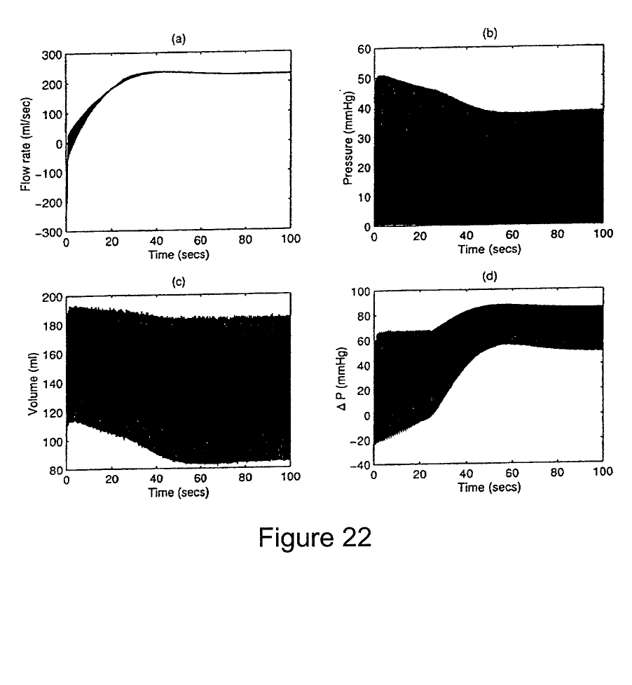

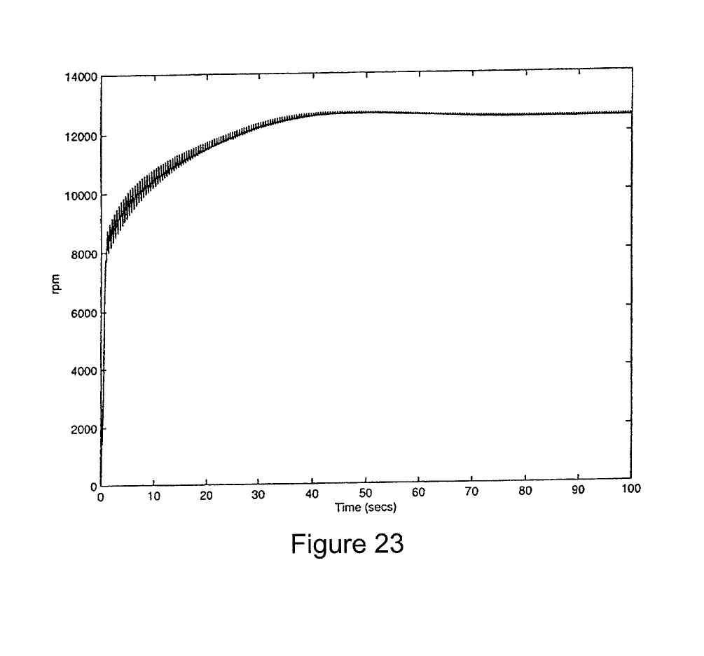

FIGS. 21, 22 and 23 show the results for the failing heart during exercise assisted by VAD with the designed controller. FIG. 21 indicates a fairly constant aortic pressure around 95 mmHg. The volume of the LH with VAD support reduced from 215/275 ml, observed without VAD assistance, to approximately 79/117 ml, a normal range. The LH pressure was reduced to about 50/10 mmHg. As shown in FIG. 22b, the RH pressure reduced to 38/0 mmHg, which is slightly above the normal range, but is a significant improvement over a fatal 49/0 mmHg without the VAD. FIG. 22a indicated no back flow through the pump and also that the average pressure head was closer to the 75 mmHg setpoint (as shown in FIG. 22d), compared to a weakened heart without a VAD. As represented in FIG. 22c, the stroke volume increased from 80 ml, a failure condition, to 101 ml which is near the stroke volume for a normal heart under exercise. FIG. 23 shows that the rpm variations were considerably reduced during the limit cycle, as well as less than the limit cycle rpm variation with healthy heart under exercise.

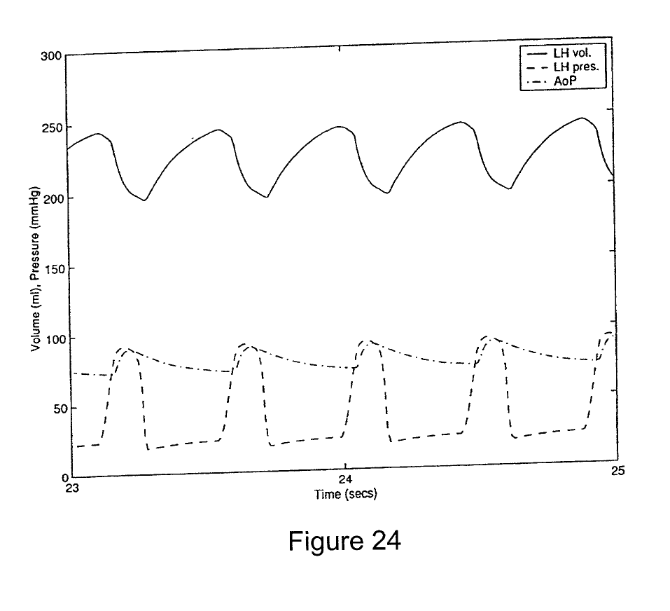

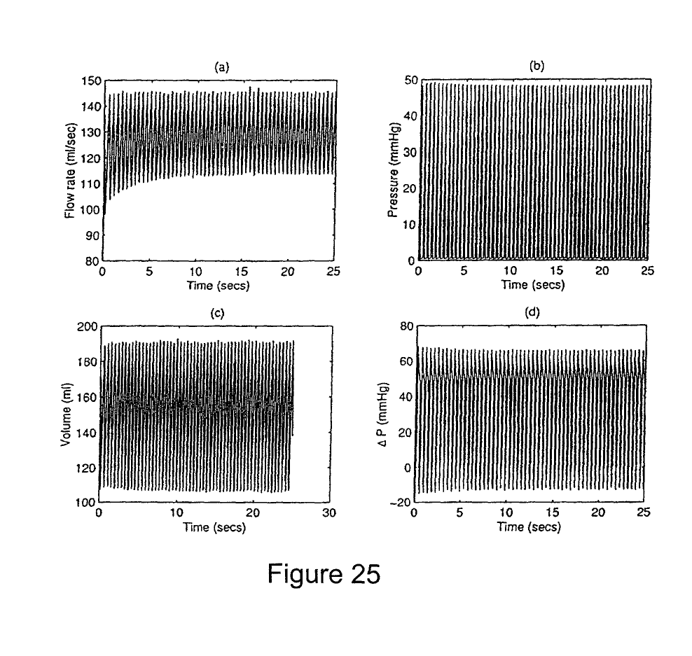

FIGS. 24 and 25 show the results for the weakened heart during exercise assisted by VAD with a constant rpm setpoint (the control strategy used). A constant rpm setpoint of 9749 rpm was selected because this was the average rpm predicted by the controller at rest. FIG. 24 indicates systolic and diastolic aortic pressures of 87/76 mmHg. The volume of the LH with VAD support at constant rpm reduced from 215/275 ml, observed without VAD assistance, to approximately 175/219 ml. However this value is higher than the normal range. As depicted in FIG. 25b, the RH pressure did not reduce significantly, which is above the normal range, and was not a significant improvement without the VAD. FIG. 25a indicates no back flow through the pump and also that the average pressure head was much less than the 75 mmHg setpoint (as shown in FIG. 25d), compared to a weakened heart with a VAD controlled by a PI controller. As illustrated in FIG. 25c, the stroke volume increased from 80 ml (a failure condition) to 88 ml, which is significantly less than the stroke volume for a normal heart under exercise, 110 ml. From FIGS. 24 and 25, it was concluded that a constant rpm setpoint is very ineffective and has a far inferior performance than the VAD controlled by the PI controller of the invention.

The LH pressure, volume, and the aortic pressure reached an almost constant value at the steady state for an asystolic left heart with VAD under exercise. Small oscillations were seen in the LH volume due to the pulsatility of the RH. The ΔP is exactly 75 mmHg, the setpoint. The RH volume and RH pressure stabilized within normal limits. Thus, the PI controller ensured that the VAD keeps the person alive and stable during exercise, even with an asystolic LH, provided the cardiac demand is within its design limits.

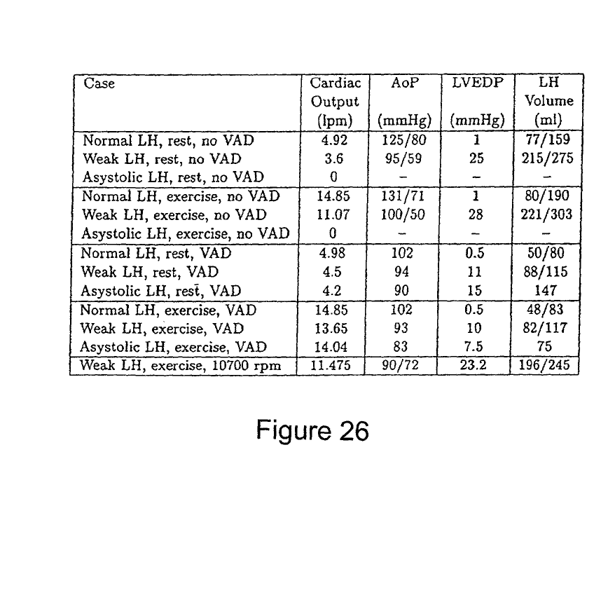

FIG. 26 compares cardiac output, AoP, the left ventricular end diastolic pressure (LVED-P) and LH volume for the conditions under which the Example was performed. FIG. 26 illustrates that the VAD reduced the LVEDP and increased the cardiac output to near normal.

Based on the information from this Example, further iterations to the model and the controller design can be made. Such iterations would improve the model and the controller design that incorporates the model.

Thus, as described above, maintaining an average pressure difference by a PI controller between left heart and aorta provides an effective way to control the LVAD with the natural heart over a wide range of conditions. The PI controller offers a quick settling time and very low flow oscillations. This advantage is possible because maintaining the prescribed pressure differential synchronizes the assist and natural perfusion, thus indirectly incorporating natural cardiovascular regulation into the VAD control. The proposed control objective thus reflects the physiological demands of perfusion and is simple enough to allow for simple control laws, resulting in better device efficacy and reliability. Though the simplicity of the controller comes at the cost of two additional pressure sensors, the number of controllers can be reduced further by estimating the pressure differential using intrinsic pump parameters.

Having described these aspects of the invention, it is understood that the invention defined by the appended claims is not to be limited by particular details set forth in the above description, as many apparent variations thereof are possible without departing from the spirit or scope thereof.

We claim:

1. A method for pumping a body fluid, comprising:
providing a pump for the body fluid;
determining the pressure differential across the pump; and
maintaining the pressure differential at a substantially constant value.

2. The method of claim 1, further comprising determining the pressure differential by measuring the pressure at the input and output of the pump.

3. The method of claim 2, including measuring the pressure at the input and the output using pressure sensors.

4. The method of claim 1, further comprising determining the pressure differential by estimating the input and output pressure of the pump using the operating parameters of the pump.

5. The method of claim 1, including maintaining the pressure differential using a control system.

6. The method of claim 5, wherein the control system contains a feedback controller for maintaining the pressure differential.

7. The method of claim 1, wherein maintaining the pressure differential provides an adequate flow of the body fluid under different pathological conditions.

8. The method of claim 1, wherein the body fluid is blood and the pump is a ventricular assist device.

9. A method for controlling a pump for a body fluid, comprising: providing a pump for the body fluid;
determining the pressure differential across the pump; and
maintaining the pressure differential at a substantially constant value.

10. The method of claim 9, further comprising determining the pressure differential by measuring the pressure at the input and output of the pump using pressure sensors.

11. The method of claim 9, further comprising determining the pressure differential by estimating the input and output pressure of the pump using the operating parameters of the pump.

12. The method of claim 9, including maintaining the pressure differential using a control system containing a feedback controller.

13. The method of claim 9, wherein the body fluid is blood and the pump is a ventricular assist device.

14. A method for controlling a pump for a body fluid, comprising:
providing a pump for the body fluid;
determining the pressure differential across the pump; and
maintaining the pressure differential at a substantially constant value, thereby providing an adequate flow of the body fluid under different pathological conditions.

15. A method for controlling a pump for a body fluid, comprising:
providing a pump for the body fluid;
measuring the average pressure differential across the pump by using pressure sensors; and
maintaining the average pressure differential at a substantially constant value, thereby providing an adequate flow of the body fluid under different pathological conditions.

16. A system for pumping a body fluid, comprising:
a pump;
means for determining the pressure differential across the pump; and
means for maintaining the pressure differential at a substantially constant value.

17. The system of claim 16, wherein the determining means include means for measuring the input and output pressure of the pump.

18. The system of claim 17, wherein the measuring means includes pressure sensors.

19. The system of claim 16, wherein the determining means includes the operating parameters of the pump.

20. The system of claim 16, wherein the maintaining means includes a control system functioning to adapt the flow generated by pump the to the changing physiological requirements of the body.

21. The system of claim 20, wherein the control system contains a feedback controller.

22. The system of claim 16, wherein the body fluid is blood and the pump is a ventricular assist device.

23. A system for pumping a body fluid, comprising:
a pump; and
a control system for maintaining a physiologically-sufficient flow of the body fluid through the pump, the control system maintaining a pressure differential across the pump to ensure adequate flow despite the changing conditions of the body fluid.

24. The system of claim 23, wherein the control system contains a feedback controller.

25. The system of claim 23, wherein the body fluid is blood and the pump is a ventricular assist device.

26. A system for a body fluid, comprising:
a pumping system for pumping the body fluid; and
a control system for maintaining a physiologically-sufficient flow of the body fluid through the pumping system, the control system maintaining a pressure differential across the pumping system to ensure adequate flow despite the changing conditions of the body fluid.

27. The system of claim 26, wherein the pumping system contains a pump.

28. The system of claim 26, wherein the control system contains a feedback controller.

29. The system of claim 26, wherein the body fluid is blood and the pump is a ventricular assist device.

30. A method for controlling the flow of a body fluid, comprising:
providing a pump in the flow of the body fluid;
determining the pressure differential across the pump; and
maintaining the pressure differential at a substantially constant value.
